(19) United States
(12) Patent Application Publication
(10) Pub. No.: US 2016/0015510 A1
(43) Pub. Date: Jan. 21, 2016

(54) Lock Block Tendon Anchor

(71) Applicant: John P Fulkerson, Litchfield, CT (US)

(72) Inventor: John P Fulkerson, Litchfield, CT (US)

(21) Appl. No.: 14/796,019

(22) Filed: Jul. 10, 2015

Related U.S. Application Data
(60) Provisional application No. 62/025,280, filed on Jul. 16, 2014.

(51) Int. Cl. A61F 2/08; A61B 17/04

(52) U.S. Cl. CPC A61F 2/0811; A61B 17/0401; A61B 2017/0448; A61F 2002/0817; A61F 2002/0864; A61F 2002/0858; A61B 2017/0409; A61F 2002/0882

(57) ABSTRACT

A lock block anchor provides secure fixation of a tendon, ligament, synthetic material or strand of any sort into a tunnel, slot or socket, in bone for orthopedic uses. The tendon, ligament or synthetic material is attached to the exterior of a roughened surface on the anchor and extends beyond the distal end of the anchor. When the anchor is locked into place following suitable preparation of the bone socket, it compresses to the tendon, ligament or synthetic material against the under surface of adjacent cortical bone. The compression and additional length promote faster, more effective healing.

### BACKGROUND OF THE INVENTION

The present disclosure relates to a method and apparatus for securing a tendon, or other soft tissue, into a subcortical hole, tunnel, or socket in bone for orthopedic uses.

### SUMMARY OF THE INVENTION

A lock block anchor constructed in accordance with the invention provides secure fixation of a tendon, ligament, synthetic material or strand of any sort into a tunnel, slot or socket, in bone for orthopedic uses. This anchor is unique in part because it allows for a length of the tendon, ligament or synthetic material extending beyond the distal end of the anchor, to be placed into and locked within the bone under compression to the under surface of adjacent cortical bone following suitable preparation of the bone socket. The compression and additional length should promote faster, more effective healing.

The tendon, ligament or other material is attached to the lock block anchor eccentrically towards its distal end, such that once the anchor is introduced into the hole or slot, the anchor is tilted within the bone sub-cortically and then the top, proximal end of the anchor is deployed under the cortex or edge of bone to create a permanent attachment of the tendon, ligament or other material under compression within the bone.

The anchor itself is desirably narrowest towards the first introduced tip, with the tendon, ligament or synthetic material secured to the exterior, top surface of the lock block anchor distally (not within the anchor as in Supinski U.S. Pat. No. 6,875,214) and eccentrically, such that the attached tissue extends beyond the end of the anchor to maximize tissue within the bone for optimal fixation under compression upon deployment. The emphasis here is on maximizing tissue within the socket by attaching it outside the anchor and providing secure compressive loading of the tissue within the bone upon deployment of the anchor under the bone cortex by virtue of the implant's unique deployment design. A serrated grid or roughened surface for additional grip of the tendon, ligament or synthetic material to the anchor can be added to minimize or eliminate slippage on the anchor. It is desirable to have some of the attached tissue extending beyond the tip of the anchor to maximize tissue within the bone, unlike other patented anchor designs noted to date. For a tendon graft in bone, approximately one centimeter of tendon beyond the tip is desirable but not necessary for increased healing surface within bone.

If sutures are used to attach the graft on the lock block, they may be left long such that they, along with the tendon, ligament or synthetic material being attached, are pulled up through the hole, along with the tendon (or ligament or synthetic material), while simultaneously deploying the proximal end of the anchor under the cortex in order to toggle the lock block anchor and secure the tendon (or ligament or synthetic material) into the hole under the cortex.

A delivery device is removably attached to the proximal end of the anchor to introduce the anchor and attached tendon into the hole in a longitudinal orientation. The hole should be large enough to allow easy introduction of the lock block/tendon graft into the subcortical hole. Once the anchor is fully beneath the cortical edge, the delivery device is detached and used to press against the proximal end of the anchor and rotate it under the cortex by pushing it securely under the edge while pulling up on the tendon to toggle the anchor. Since there is cancellous bone under cortex, the deployment requires firm compression into the cancellous subcortical bone. A tapping mechanism might be used to fashion a trough in the sub-cortical area to allow for anchor toggle and secure displacement under the cortex. An angled curette or alternate custom designed tool for this purpose should be used to access and assure adequate subcortical space for lock block deployment.

### BRIEF DESCRIPTION OF THE DRAWINGS

An exemplary embodiment of carrying out the invention is described herein below with reference to the following figures.

FIG. 1 is a perspective view of the anchor and the delivery device of an exemplary embodiment of the invention attached in a coaxial configuration for insertion into a subcortical hole in bone;

FIG. 2 is a close-up perspective view of the embodiment shown in FIG. 1, wherein the anchor and the delivery device are detached for exerting force to rotate the anchor so that its proximal end is underneath the cortex surrounding the subcortical hole and the tendon is compressed against subcortical bone on the other side of the hole.

### DETAILED DESCRIPTION OF AN EXAMPLARY EMBODIMENT

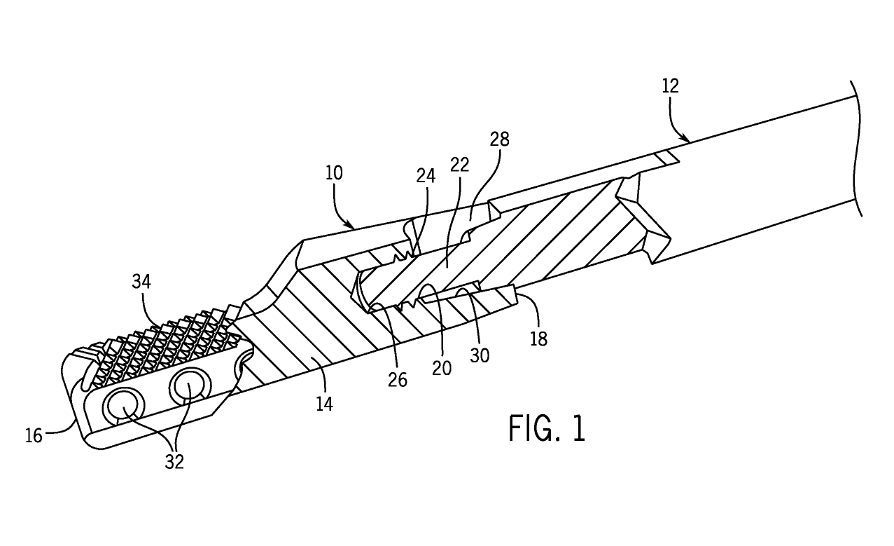

Referring to FIG. 1, the apparatus is generally comprised of a lock block anchor 10 and a delivery device 12. The lock block anchor 10 has an elongated body 14 and a distal end 16 and a proximal end 18. A serrated grid 20 is provided on at least one surface on the anchor 10 near the distal end 16, and serves as a tendon attachment zone. It is desired that the distal end 16 of the anchor 10 be narrower than the proximal end 18. In other words, the plane in which the serrated grid lies is recessed with respect to the outer surface of the proximal end 18 of the anchor 10. The recess accommodates the tendon which is sutured to the serrated grid 34. This configuration is advantageous for inserting the lock block anchor 10 into a subcortical hole in a bone (not pictured), as the distal end 16 is the first end of the anchor to be inserted into the bone. The anchor 10 includes suture holes 32 extending through the lock block anchor 10 underneath the serrated grid surface 34. The tendon (or other tissue or synthetic material) is laid longitudinally over the length of the anchor 10 with the tendon over the serrated grid 34, and desirably overhanging the distal end 16 of the anchor by about 1 cm. In the embodiment shown in FIG. 1, the tendon is sutured to anchor by passing the sutures through the suture holes 32 underneath the grid 34. As mentioned above, the sutures should be left long to facilitate final placement of the anchor 10.

Other suitable means of attaching the tendon (or other tissue or synthetic material) to the anchor 10 over the serrated grid 34, besides suturing, may be used such as clamps, screws, meshes, tightening sleeves or other methods known to those skilled in the art. If alternative means are used, it may be desirable to modify the anchor 10 to accommodate the alternative means of attachment.

The insertion of the anchor 10 into the bone is substantially in the direction of a longitudinal insertion axis. In FIG. 1, the delivery device 12 is shown to be attached to the anchor 12 in a coaxial orientation so that a longitudinal force applied to the delivery device inserts the lock block anchor 10 longitudinally into a hole prepared in the bone. FIG. 1 depicts a delivery finger 22 with screw threads 24 that is used to removeably attach the delivery device 12 to screw threads 20 in an aperture 26 adjacent an installation cavity 28 on the proximal end 18 of the anchor 10.

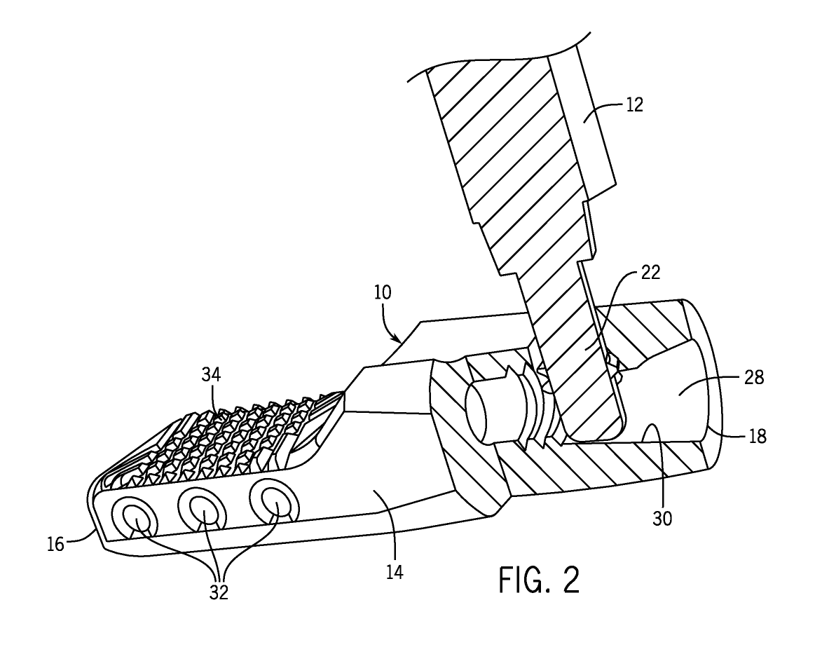

FIG. 2 depicts the delivery device 12 detached from the anchor 10 and pushing against a shoulder 30 on the inside surface of an installation cavity 28 on the proximal end 18 of the anchor. This depiction illustrates the orientation after the anchor 10 has been rotated partially and the delivery device is being used to push down through the subcortical hole in the bone and rotate the anchor 10 further from the longitudinal orientation of the hole in the bone. While the preferred application of the invention is to permanently fix tendon to bone, the invention can be used to fix other soft tissue such as ligament or synthetic material into a bone for any type of orthopedic or other application. In general, the method comprises a first step of eccentrically placing the tendon (not shown) on the serrated grid 34 and suturing the tendon to the serrated grid 34 using suture holes 34 or otherwise attaching the tendon to the serrated grid 34. Next, the delivery finger 22 on the delivery device 12 is inserted into the cavity 28 in the proximal end 18 of the lock block anchor 10 and screwed into the screw threads 24 on the anchor 10. The lock block anchor 10 is then inserted, distal end first, into the pre-drilled and pre-prepared hole in the bone (not shown) by imparting longitudinal movement to the delivery device 12 and attached anchor 12. Once the anchor 12 has been inserted into the bone, the delivery finger 22 is unscrewed from the anchor 10 and removed. Finally, a transverse force is applied to the shoulder 30 on the anchor 10 by the delivery finger 22 and the tendon is pulled upward through the hole in the bone to position the proximal end 18 of the anchor 10 under the cortex surrounding the hole in the bone and compress the tendon secured onto the serrated grid 34 of the anchor 10 against the bone on the other side of the hole.

In general, it should be understood that while the Figures depict the lock block anchor and delivery device to have certain shapes, alternate shapes may also be suitable. Further, it should be understood that the suture holes need not be substantially cylindrical, and need not pass entirely through the lock block anchor. For example, notches on the opposing side of the anchor may be used to hold the sutures in place. Also, as mentioned, other suitable means of attaching the tendon (or other tissue or synthetic material) to the anchor 10 over the serrated grid 34, besides suturing, may be used such as clamps, screws, meshes, tightening sleeves or other methods known to those skilled in the art. If such alternative means of attaching are used, it may be desirable to modify the anchor 10 to accommodate the alternative means of attachment.

A lock block anchor provides secure fixation of a tendon, ligament, synthetic material or strand of any sort into a tunnel, slot or socket, in bone for orthopedic uses. The strand is secured to the exterior of the anchor over a roughened attachment zone such that a length of the strand extends beyond the distal end of the anchor. When properly locked into a suitably prepared hole in the bone, the anchor compresses the strand to the under surface of adjacent cortical bone. The compression and additional length should promote faster, more effective healing.

What is claimed is:

1. An apparatus for securing a strand into a subcortical hole in bone for orthopedic uses, the apparatus comprising:
a lock block anchor having an elongated body with a longitudinal insertion axis and a distal end and a proximal end;
a strand attachment zone on at least a portion of the surface near the distal end of the elongated body, the strand attachment zone being adapted to receive a strand laid substantially longitudinally along the surface of the elongated body and enable attaching the strand against the strand attachment zone and securing the strand eccentrically on the elongated body; and
an installation cavity having a shoulder on the proximal end of the elongated body; and
a delivery device comprising an elongated rod adapted to be removeably fixed in longitudinal alignment with the elongated body of the anchor by removeably securing the rod within the installation cavity on the proximal end of the anchor, whereby the delivery device is used to place the anchor within a subcortical hole in the bone with the strand longitudinally and eccentrically attached, and is further used to assist rotation of the placed anchor to a permanent orientation and compress the strand against subcortical bone by pressing the rod against the shoulder of the installation cavity on the proximal end of the elongated body of the anchor such that the proximal end of the anchor is rotated underneath cortex surrounding the subcortical hole in the bone.

2. The apparatus as recited in claim 1 wherein the strand consists of one of a tendon, a ligament, and synthetic material.

3. The apparatus as recited in claim 1 wherein the lock block anchor further comprises means for holding the strand on the elongated body of the anchor.

4. The apparatus as recited in claim 3 wherein said means for holding comprises sutures and the apparatus further comprises at least one suture hole extending through the lock block anchor underneath the strand attachment zone.

5. The apparatus as recited in claim 1 wherein the strand attachment zone comprises a serrated grid for gripping a strand attached to the anchor.

6. The apparatus as recited in claim 1 wherein the strand attachment zone resides on a substantially planar surface that is recessed relative to an outer surface of the anchor near its proximal end.

7. The apparatus as recited in claim 1 wherein:
the installation cavity on the anchor includes a threaded finger-receiving aperture; and
the delivery device includes a threaded delivery finger;
whereby the delivery finger can be attached in longitudinal alignment to the finger receiving aperture through the proximal end of the anchor in order to apply force on the delivery device along the longitudinal insertion axis of the lock block anchor for insertion into the subcortical hole in the bone, and the delivery finger can be detached from the finger receiving aperture in the anchor and used to apply force on the shoulder of the anchor to rotate the proximal end of the anchor underneath the cortex of the bone surrounding the subcortical hole.

8. A method for fixing a strand to a bone comprising the steps of:
preparing a subcortical hole in a bone;
providing a lock block anchor having: an elongated body with a longitudinal insertion axis and a distal end and a proximal end; a strand attachment zone on at least a portion of the surface near the distal end of the elongated body, the strand attachment zone being adapted to receive a strand laid substantially longitudinally along the surface of the elongated body and enable attachment of the strand against the strand attachment zone and securing the strand eccentrically on the elongated body; and an installation cavity having a shoulder on the proximal end of the elongated body placing a tendon longitudinally along the anchor on tendon attachment zone;
attaching the strand to the anchor so that it is secured eccentrically to the anchor;
removably attaching a delivery device to the proximal end of the lock block anchor;
inserting the lock block anchor, distal end first, longitudinally into the subcortical hole in the bone by applying a force on the delivery device along the longitudinal insertion axis of the anchor;
detaching the delivery device from the lock block anchor;
pressing the delivery device against the shoulder of the installation cavity on the proximal end of the anchor to rotate the anchor and position the proximal end of the anchor underneath the cortex surrounding the subcortical hole and compress the strand against the subcortical bone on the other side of the subcortical hole; and
removing the delivery finger from the subcortical hole leaving the anchor in a rotated position under the cortex and the stand extending from the anchor through the subcortical hole and substantially compressed into the subcortical cavity.

9. The method of claim 8 wherein the strand is placed longitudinally along the anchor on the strand attachment zone such that the end of the strand overhangs the distal end of the anchor.

10. The method of claim 9 wherein the strand consists of one of a tendon, a ligament, and synthetic material.
